(19) United States
(12) Patent Application Publication
(10) Pub. No.: US 2012/0106716 A1
(43) Pub. Date: May 3, 2012

(54) METHOD AND EQUIPMENT FOR AN X-RAY APPARATUS

(75) Inventor: Lars Herrnsdorf, Lindome (SE)

(73) Assignee: RTI ELECTRONICS AB, MOLNDAL (SE)

(21) Appl. No.: 13/320,102

(22) PCT Filed: May 12, 2009
(86) PCT No.: PCT/SE09/50530

§ 371 Date: Dec. 13, 2011

(51) Int. Cl. A61B 6/08

(52) U.S. Cl. 378/205

(57) ABSTRACT

A method for determining alignment of light and an X-ray fields of an X-ray apparatus, comprising: directing the light field onto an exposure area, positioning a scale and an X-ray indicating element in association with each other at the exposure area such that said scale and X-ray indicating element cross an edge of the light field, wherein said X-ray indicating element emits light upon exposure to X-rays so that parts exposed to X-rays can be distinguished from non-exposed parts, determining a position on the scale where the light field edge is positioned, and directing the X-ray field onto the exposure area. The method comprises generating an image of the scale and the X-ray indicating element using a digital camera, determining a position on the scale where an edge of the X-ray field is positioned, and comparing the scale positions of the edges of the light and X-ray fields.

### TECHNICAL FIELD

This invention relates to a method and equipment for determining alignment of a light field and an X-ray field of an X-ray apparatus.

### BACKGROUND OF THE INVENTION

Checking of the actual distribution of an X-ray field in comparison to a set value is an important and legally regulated test for X-ray equipment used in e.g. radiography, mammography and therapy. Significant discrepancies between actual and presumed X-ray field distributions may result in additional exposure to X-rays and unnecessary doses.

It is common that an X-ray equipment is provided with a light source that is arranged to produce a light field that has a similar distribution as the X-ray field. This light field can be used instead of the X-ray field when adjusting e.g. the area to be exposed or the position of a patient.

To make use of such a light field it is important that the light and X-ray fields are well aligned with each other. For this reason it is important that the equipment is calibrated, which calibration involves the use of suitable means and methods for determining to what extent the two fields coincide. In such a determination it is needed to somehow visualize the X-ray field such as to allow comparison of the two fields.

Conventionally, X-ray films have been used to determine the distribution of the X-ray field. To avoid the time and effort associated with the development of such films some alternative devices have been presented.

In one example, the device is provided with an afterglowing phosphor screen that visualises the X-ray radiation field. To determine the deviation between the two fields the device is initially adjusted to the light field according to certain marks. After exposing the device to X-ray the afterglow shows the size and position of the X-ray field. A scale on the device shows the magnitude of any deviation from the light field.

US 2006/0285646 shows another example wherein a device in the form of an X-ray “ruler” is provided with a scale in the form of a row of X-ray sensors connected to a corresponding light-emitting element. This device is intended to be placed at an edge and half-way into the light field. When exposed to the X-ray field the light-emitting elements show where the edge of the X-ray device was positioned during exposure.

Although the type of devices exemplified above has simplified the procedure compared to the use of X-ray films, there is still a need for improvements in this technical area.

### SUMMARY OF THE INVENTION

An object of this invention is to provide a method and equipment that allows for a more efficient procedure for determining alignment of light and X-ray fields of an X-ray apparatus. This object is achieved by the method, arrangement and device defined by the technical features contained in independent claims 1, 6 and 11. The dependent claims contain advantageous embodiments, further developments and variants of the invention.

The invention concerns a method for determining alignment of a light field and an X-ray field of an X-ray apparatus, comprising the steps of: directing the light field onto an exposure area; positioning a scale and an X-ray indicating element in association with each other at the exposure area such that said scale and X-ray indicating element cross an edge of the light field, wherein said X-ray indicating element is configured to emit light upon exposure to X-rays in such a way that parts exposed to X-rays can be distinguished from non-exposed parts; determining a position on the scale where the light field edge is positioned; and directing the X-ray field onto the exposure area.

The inventive method comprises the steps of: generating an image of the scale and the X-ray indicating element when the X-ray indicating element emits light due to the exposure of said X-ray field using a digital camera; determining a position on the scale where an edge of the X-ray field is positioned by analyzing said image; and comparing the scale positions of the edges of the light and X-ray fields.

An advantageous effect of this method is that it allows for a simple procedure since digital images are easy to handle and easy to analyze on a computer screen. Further, the method is flexible since analysis can be carried out at a later stage and in another location as digital images are easy to store and transfer. In addition, the inventive method provides for a very rapid and easy documentation of the results in that the image is quickly and easily stored.

In a preferred embodiment of the invention the step of generating the image comprises the steps of: recording the X-ray exposure with a digital video camera; and selecting an image from the video recording. A video camera can be started before and stopped after the X-ray exposure and digital video recordings allows for analysis image by image. Thereby it is possible to select a proper image without any need for synchronizing the trigging of the camera with the X-ray exposure.

Analyzing the image by viewing the image on a computer screen using image/video processing software has the advantage that no complicated algorithms that take account of different camera positions are needed, as is the case when using computerized procedures for analyzing the image. The camera can thereby be relatively freely adjusted.

The invention also concerns an arrangement for determining alignment of a light field and an X-ray field of an X-ray apparatus, said arrangement comprising an X-ray indicating element configured to emit light upon exposure to X-rays in such a way that parts exposed to X-rays can be distinguished from non-exposed parts, and a scale arranged in association with the X-ray indicating element, wherein said element and scale are configured to allow placement on an exposure area for said light and X-ray fields such as to allow determination of where on the scale an edge of the light field and an edge of the X-ray field are positioned when said fields are directed onto said exposure area. The inventive arrangement comprises a digital camera arranged to generate an image of the scale and the X-ray indicating element when the X-ray indicating element emits light due to an exposure of said X-ray field.

The invention also concerns a device for determining alignment of a light field and an X-ray field of an X-ray apparatus, said device comprising an X-ray indicating element configured to emit light upon exposure to X-rays in such a way that parts exposed to X-rays can be distinguished from non-exposed parts, and a visible scale arranged in association with the X-ray indicating element such as to allow determination of where on the scale an edge of the light field and an edge of the X-ray field are positioned. The inventive device comprises a first and a second unit, wherein each of said units comprises a set of said X-ray indicating element and scale, and wherein said first and second units are rotatably connected to each other.

Such a device has the advantage that it can be positioned at two edges of X-ray fields of different size and possibly four edges depending on the particular design. A fewer number of X-ray exposures are thereby needed to cover all edges compared to single units. Further, it takes a minimum of space when folded together.

In a preferred embodiment of the inventive device it comprises four units, each of which having an elongated shape and comprising a set of said X-ray indicating element and scale, wherein said four units are rotatably connected to each other such as to allow formation of a cross and to allow positioning on top of each other. Such a device can be adapted to most detection areas and dispenses with the need for a detection screen that covers the entire detection area. Such screens are relatively expensive and are not easy to carry around.

### BRIEF DESCRIPTION OF DRAWINGS

In the description of the invention given below reference is made to the following figure, in which:

FIG. 1 shows, in an exploded perspective view, a unit for determining alignment of a light field and an X-ray field of an X-ray apparatus,

FIG. 2 shows a composite device for determining alignment of a light field and an X-ray field of an X-ray apparatus,

FIG. 3 shows a camera with supporting for use in the inventive method and arrangement,

FIG. 4 shows an X-ray apparatus,

FIG. 5 shows light and X-ray fields of the X-ray apparatus, and

FIG. 6 shows a magnified view of a part of FIG. 5.

### DESCRIPTION OF EXAMPLE EMBODIMENTS OF THE INVENTION

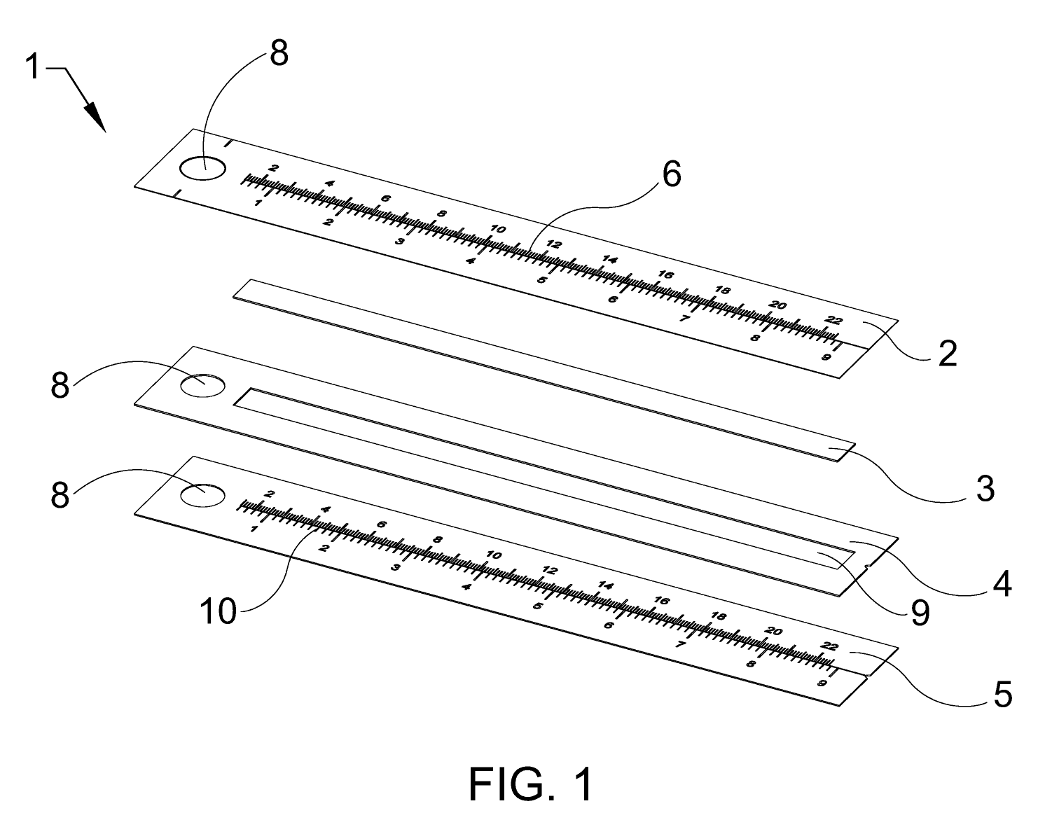

FIG. 1 shows, in an exploded perspective view, a unit 1 for determining alignment of a light field and an X-ray field of an X-ray apparatus. The unit 1 comprises an upper part 2, a mid part 4 and a lower part 5 arranged together in a layered structure. A hole 8 goes through an end part of the unit 1.

A scale 6 that extends in a longitudinal direction of the unit 1 is visibly provided on the upper part 2. The scale 6 shows both units of meters and inches with marks/scale divisions for millimeters as well as for tenths of inches.

Roughly, the alignment determination unit 1 has a flat and elongated shape, similar to a regular ruler.

The bottom part 5 is provided with an X-ray detectable scale 10 that has similar marks/scale divisions as the upper scale 6. The two scales 6, 10 are horizontally adjusted in relation to each other such as to be vertically aligned.

The bottom part 5 is made in a similar way as a printing wiring board (PWB) where the scale 10 is made in cupper. Cupper absorbs and scatters X-rays to a high extent which makes the lower scale 10 more or less opaque to, and thereby detectable by, X-rays. Other materials and elements, e.g. lead, are also well known to be detectable by X-rays.

The mid part 4 comprises a cutout 9 adapted to receive an X-ray indicating element 3 that extends along, in this case below, the upper visible scale 6 of the unit 1. The function of the mid part 4 is mainly to work as a spacer and to hold the X-ray indicating element 3 in place. Alternatively, it is possible to let the X-ray indicating element 3 constitute the entire mid part 4. A further function of the mid part 4 is to provide a suitable background to at least a part of the scale 6.

The X-ray indicating element 3 is in this example a layered flat unit that fluorescents upon exposure to X-rays, i.e. it emits light when subjected to an X-ray field. Various light-emitting X-ray indicators are commercially available. In this case the X-ray indicating element 3 comprises Gd2O2S:Tb which makes the indicator 3 sensitive and capable of emitting reasonably large amounts of light even when subjected to a relatively short and weak X-ray exposure. This is an advantage when using a camera (see below) for producing an image of the emitting of light and also makes it possible to use clinically relevant settings of an X-ray apparatus during alignment determination. Further, the exemplified indicating element 3 emits light during a rather short period of time, less than 1 ms, after termination of X-ray exposure, which is an advantage for dynamic X-ray distribution studies. A further advantage of the X-ray indicating element 3 used here is that it is flexible.

Various types of fluorescent, phosphorescent or electronic (e.g. X-ray sensor+light emitting diode) X-ray indicating elements are however possible to use for the principle of the invention. Important is that visible light is emitted upon exposure to X-rays and that the indicating element has a reasonable extension length in at least one dimension such that it can be placed across an edge of an X-ray field.

The X-ray indicating element 3 is configured to emit light upon exposure to X-rays in such a way that parts exposed to X-rays can be distinguished from non-exposed parts. This means that the element 3 when placed across an edge of an X-ray field will exhibit a boundary line during and shortly after X-ray exposure which boundary line corresponds to the edge of the X-ray field and divides the element 3 into a light-emitting exposed part and a non-exposed part that does not emit light. The X-ray indicating element 3 described here has a high resolution which means that the boundary line becomes as thin and sharp as possible. How the X-ray indicating element 3 is used in the alignment determination is further described below.

Main functions of the upper part 2 are to work as a carrier for the upper scale 6 and to protect the underlying X-ray indicating element 3. The upper part 2 is made of a plastic material and is transparent to allow light emitted from the X-ray indicating element 3 to pass through.

The whole alignment determination unit 1 is flexible which e.g. is advantageous if to be used on a rounded surface, such as a phantom.

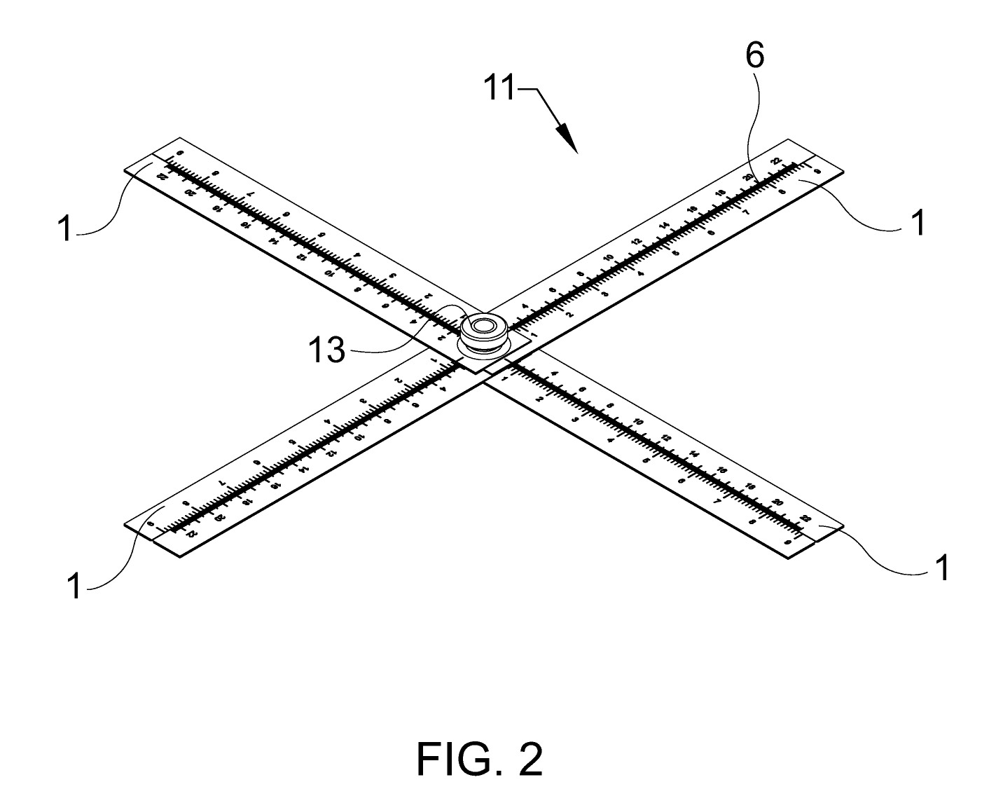

FIG. 2 shows a composite device 11 for determining alignment of a light field and an X-ray field of an X-ray apparatus according to the invention. This composite device 11 is a combined set of, in this particular example, four alignment determination units 1 hold together by a hub member 13 placed in the hole 8 of each unit 1. The individual units 1 are rotatably connected to each other such as to allow formation of a cross, as shown in FIG. 2, and to allow positioning on top of each other such as to take up less space and be easier to carry around. The cross formation shown in FIG. 2 is useful for placement across all four edges of a rectangular light or X-ray field.

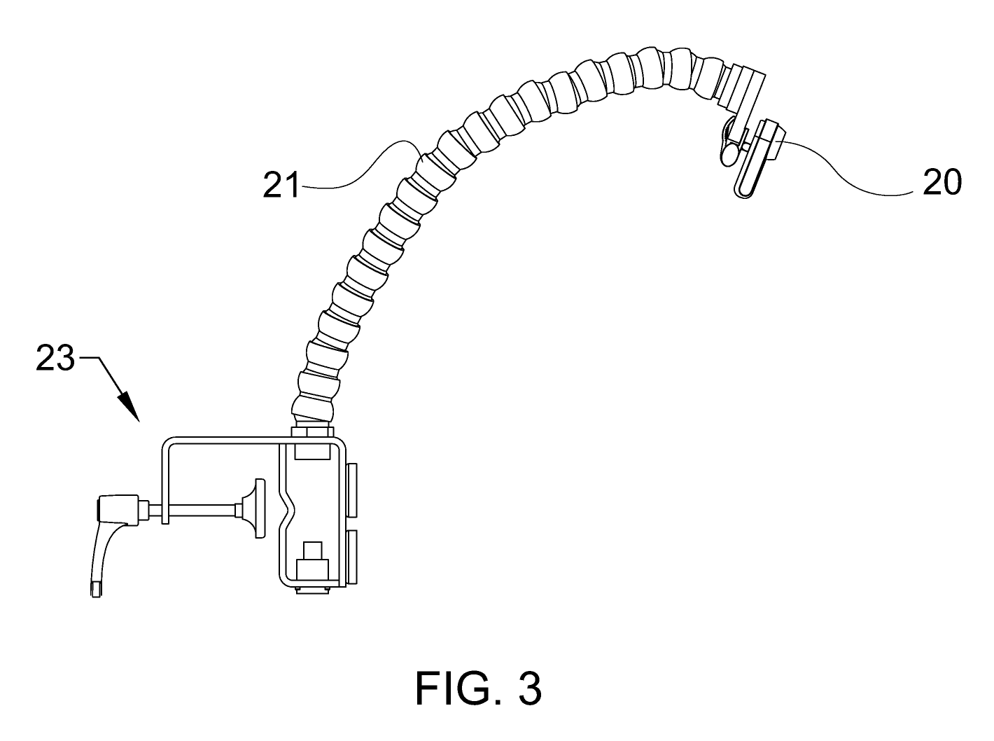

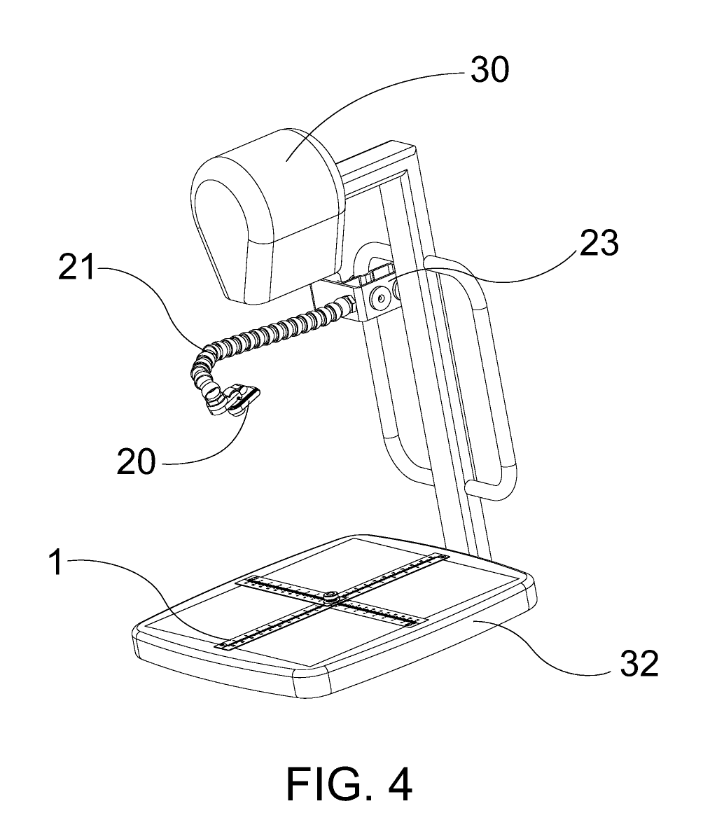

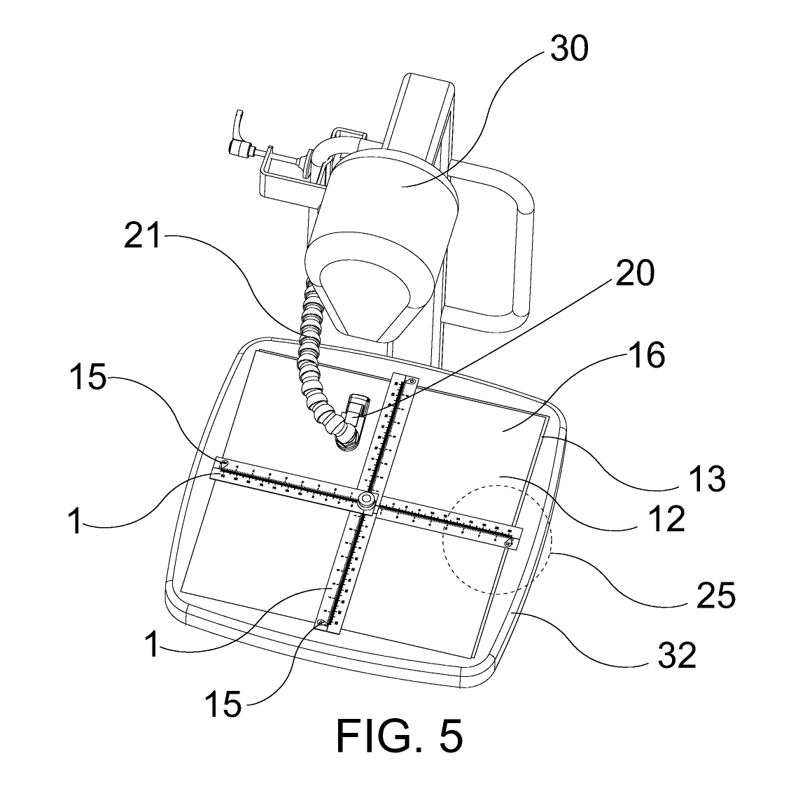

FIGS. 3-5 show a camera 20 arranged on a flexible arm 21 that is connected to a fastening arrangement 23 for fastening the arm 21 and camera 20 to an X-ray apparatus 30, as shown in FIGS. 4 and 5, or to something else near to the X-ray apparatus 30. FIGS. 4-5 further show an alignment determination unit 1 that has been positioned onto an exposure area 16 that forms an upper surface of a digital X-ray screen detector 32. The alignment determination unit 1 forms in this example part of a composite device 11 as shown in FIG. 2. The X-ray apparatus 30 shown in FIGS. 4 and 5 is capable of generating both a light field 13 and an X-ray field 12 for direction onto the exposure area 16 (see FIG. 5).

The flexible arm 21 makes it easy to adjust the camera 20 to different X-ray apparatuses and to different settings of a certain apparatus. In particular it is important to avoid that the camera 20 blocks the X-rays. The camera 20 is in this case adjusted such that its field of view covers the entire exposure area 16.

The camera 20 is a high-resolution digital video camera that works with the light of the visible spectrum. This means e.g. that it is capable of recording a relatively high number of digital images per second. The camera 20 is connectable (via cable or wire-less) to a computer (not shown) for e.g. storing, viewing, processing and analyzing of video clips and individual images recorded.

FIG. 5 shows both the light field 13 and the X-ray field 12 directed onto the exposure area 16. As indicated in FIG. 5, the X-ray field 12 is slightly displaced in relation to the light field 13. Markers 15 have been positioned on each of the alignment determination units 1 such as to indicate the position of the light field 13 on the upper, visible scale 6. The markers 15 are made of steel such as to be at least partly opaque to X-rays and thereby be detectable by X-rays.

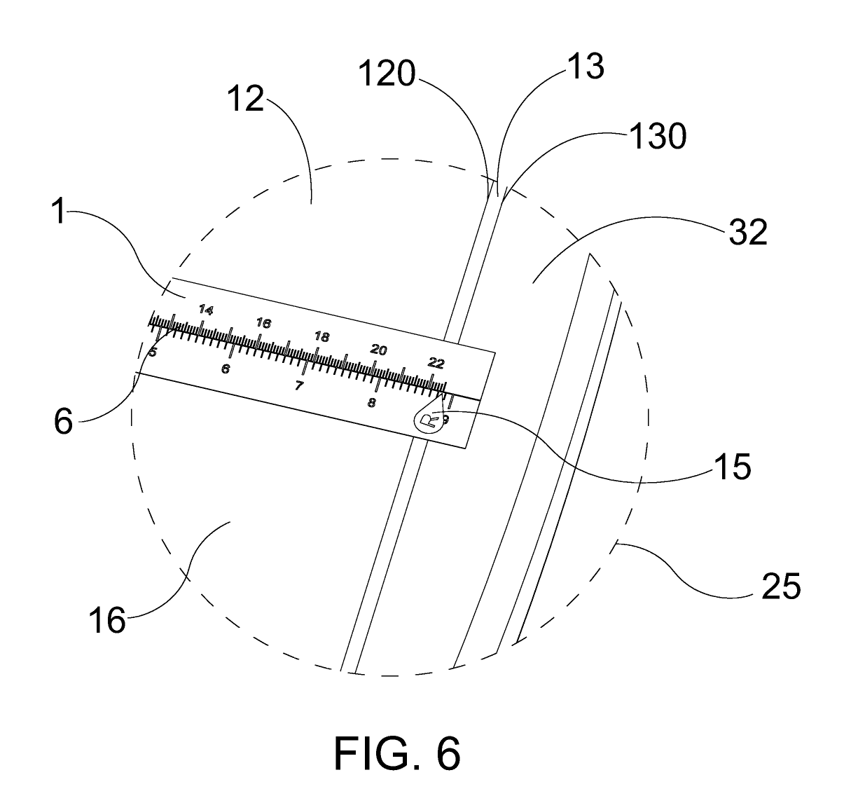

The displacement and distorsion, i.e. the lack of alignment, of the light and X-ray fields 13, 12 indicated in FIG. 5 is more clearly shown in FIG. 6 which shows a magnified view of a part 25 of FIG. 5 (indicated with a dashed line in FIG. 5). As can be seen in FIG. 6, an edge 120 of the X-ray field 12 is displaced somewhat to the left of an edge 130 of the light field 13. The marker 15 indicates the position on the scale 6 where the light field edge 130 is positioned.

In the following a preferred method for determining the alignment of the light field 13 and the X-ray field 12 of the X-ray apparatus 30 will be described. Initially, the steps of directing the light field 13 onto the exposure area 16 and positioning/adjusting the composite alignment determination device 11 such that each individual alignment determination unit 1 crosses an edge 130 of the light field 13 are performed.

In a following step, the position on the scale 6 where the light field edge 130 is positioned is registered on each unit 1 by placing the marker 15 such as to indicate said position on the scale 6. At this stage the light field 13 can be turned off or blocked.

In a following step recording with the digital video camera 20 is started. As described above, the camera 20 is adjusted such that its field of view covers the entire exposure area 16 which means that all X-ray and light field edges 120, 130 and all scales 6 and markers 15 are captured on the video recording.

In a following step an X-ray exposure is made by directing the X-ray field 12 onto the exposure area 16 during a certain time period typically around 100 ms. In a following step the video recording is stopped, i.e. the camera 20 is stopped or paused. The video recording is stored on a computer readable medium and transferred to a computer (not shown).

In a following step the video recording is viewed and analyzed using image/video processing software installed on the computer. The software allows image by image viewing on a screen connected to the computer. Various software suitable for this purpose are commercially available.

Storing and transferring of the video recording can be carried out in many different ways. For instance, the recording can initially be stored in a memory of the camera 20 and thereafter be transferred via cable or wire-less to a computer. A skilled person in the art is familiar with storing and transferring of video recordings.

By viewing and analyzing the recorded video clip it is possible to select one or several images (out of the plurality of images forming the video clip) of the set of alignment determination units 1 showing the X-ray indicating element 3 emitting light due to the X-ray exposure. Since parts of the X-ray indicating element 3 positioned outside of the X-ray field 12 will not emit any light, such an image will show the position on the scale 6 where the edge 120 of the X-ray field 12 is positioned, i.e. where the boundary line is positioned, with the current settings of the X-ray apparatus 30.

Generation of such an image can be made also with a single-shot digital camera but this is likely to require either automatic trigging of the camera, which is complicated, or an X-ray indicating element 3 that emits light during a much longer time period, which e.g. reduces the possibility of performing dynamic X-ray distribution studies.

By using an adequate image processing software it is possible to zoom into interesting portions of the image such as to determine the position of the X-ray field edge 120 in more detail. The camera 20 should have a sufficient resolution and be positioned sufficiently close to the exposure area 16 such that the position of the X-ray field edge 120 can be determined within 1 mm, i.e. the mm scale divisions of the scale 6 should be visible on the computer screen when analyzing the image.

At this stage it is possible to compare the scale positions of the edges 130, 120 of the light and X-ray fields 13, 12. Thereby the degree of alignment of the light field 13 and the X-ray field 12 can be determined. If the alignment is not sufficient, typically within 2 mm, the X-ray apparatus 30 is adjusted (which is a known procedure and therefore not described here).

In the preferred method a user determines the position of the edge 120 of the X-ray field 12 simply by visually viewing an image on a screen, if necessary with the aid of zooming. This task may be possible to carry out with a computer program that analyses the image and automatically identifies and calculates the position of the edge 120 in relation to the scale 6. However, such an automated method would require complicated calculation algorithms that take account of the position of the camera. Even a minor adjustment of the position of the camera that changes its distance and/or angle to the exposure area 16 would require extensive calculations to compensate for the different camera position. The preferred method eliminates these problems and allows for a relatively free positioning of the camera 20.

In order to generate a useful image of the scale 6 and the edge 120 of the X-ray field 12 it is necessary that the scale 6 is visible, i.e. that some light is present. This can e.g. be achieved by having some background light in the room where the X-ray apparatus 30 is located, by leaving the light field 13 on during X-ray exposure, or by configuring the alignment determination unit 1 such that the X-ray indicating element 3 illuminates the scale 6 when subjected to the X-ray exposure. Further, also the scale 6 itself may be configured to emit light upon exposure to X-rays. Of course, the light used to visualize the scale 6 should be limited such that the light emitted from the X-ray indicating element 3 still can be detected.

The present invention provides for a simple and thorough documentation since all digital images and video clips easily can be stored on a computer readable medium. As an alternative or complement to storing all images and video clips, values of the scale positions of the field edges 120, 130 can be stored.

The unit 1 and device 11 for determining the alignment of the light field 13 and the X-ray field 12 of the X-ray apparatus 30 can also be used in an alternative method where the digital X-ray screen detector 32 is used instead of the camera. The initial steps of this method are similar to what is described above, i.e. i) directing the light field 13 onto the exposure area 16, ii) positioning/adjusting the composite alignment determination device 11 such that each individual unit 1 crosses an edge 130 of the light field 13., and iii) registering the position on the scale 6 where the light field edge 130 is positioned is registered by placing the marker 15 such as to indicate said position on the scale 6. Also the step of turning off the light field 13 can be similar. An additional step may be to activate the digital X-ray screen detector 32.

The next step in this alternative method is to perform an X-ray exposure directing the X-ray field 12 onto the exposure area 16 during a certain time period, typically around 100 ms. This step is followed by a step including an analysis of the detector image produced by the digital X-ray screen detector 32. On this detector image the X-ray field 13, the X-ray detectable scale 10 of each unit 1, as well as each marker 15 will appear. By analyzing this detector image and comparing the positions of the field edges 120, 130 in relation to the X-ray detectable scale 10 it is possible to determine the alignment of the light field 13 and the X-ray field 12 of the X-ray apparatus 30.

In a variant of this alternative method it is carried out without using the light field 13. In such a case the alignment determination device 11 and the markers 15 are initially positioned according to certain marks on top of the detector 32.

The camera 20 can be used in combination with the variants of the alternative method in that the camera 20 can be used for registering the light field 13 and/or the image of the detector 32 (e.g. by recording an image of a monitor connected to the detector 32).

The invention is not limited by the embodiments described above but can be modified in various ways within the scope of the claims. For instance, it is not necessary for the inventive method that a composite alignment determination device 11 is used; one or several individual alignment determination units 1 may be used. Neither is it necessary that the scale 6 and the X-ray indicating element 3 are integrated into the same unit 1 such as shown in FIG. 1, although such a unit simplifies the procedure. What is important is that the scale 6 and element 3 are arranged in association with each other, preferably along each other, such that the position on the scale where an edge of the X-ray field is positioned can be determined.

Moreover, the position on the scale 6 where the light field edge 130 is positioned does not necessarily have to be registered by placing a marker but can e.g. be registered by generating an image using the camera 20. This image can then be compared to the image showing the position of the X-ray field edge 120. In principle, this registration can of course also be made by making a note, mental or physical.

Viewing and analyzing videos and images do not necessarily have to be performed in connection to the light and X-ray field exposures. The camera recordings can e.g. be stored on a portable computer or memory, and be viewed and analyzed at a later stage in a different location. Of course, recorded data can also be sent and stored using a data network, such as a local computer network. Thus, it is not necessary that the computer used to analyze the data is connected to the camera 20.

Individual alignment determination units 1 can be put together to composite devices 11 that have more or fewer than four individual units to suit different applications. For instance, a fifth shorter alignment determination unit 1 may be added that is suitable for mammography.

1. Method for determining alignment of a light field and an X-ray field of an X-ray apparatus, comprising the steps of:
directing the light field onto an exposure area,
positioning a scale and an X-ray indicating element in association with each other at the exposure area such that said scale and X-ray indicating element cross an edge of the light field, wherein said X-ray indicating element is configured to emit light upon exposure to X-rays in such a way that parts exposed to X-rays can be distinguished from non-exposed parts,
determining a position on the scale where the light field edge is positioned,
directing the X-ray field onto the exposure area,
generating an image of the scale and the X-ray indicating element when the X-ray indicating element emits light due to the exposure of said X-ray field using a digital camera,
determining a position on the scale where an edge of the X-ray field is positioned by analyzing said image, and
comparing the scale positions of the edges of the light and X-ray fields.

2. Method according to claim 1, wherein the step of generating an image of the scale and the X-ray indicating element comprises steps of:
recording the X-ray exposure with a digital video camera, and
selecting an image from the video recording.

3. Method according to claim 1, comprising a step of registering the position of the light field edge by placing a marker that indicates said position on the scale.

4. Method according to claim 1, comprising a step of registering the position of the light field edge by generating an image of the scale and the edge of the light field using said camera.

5. Method according to claim 1, wherein the step of determining a position on the scale where an edge of the X-ray field is positioned by analyzing said image comprises a step of viewing the image on a computer screen using image/video processing software.

6. An apparatus for determining alignment of a light field and an X-ray field of an X-ray apparatus, said arrangement comprising
an X-ray indicating element configured to emit light upon exposure to X-rays in such a way that parts exposed to X-rays can be distinguished from non-exposed parts, and
a scale arranged in association with the X-ray indicating element, wherein said element and scale are configured to allow placement on an exposure area for said light and X-ray fields such as to allow determination of where on the scale an edge of the light field and an edge of the X-ray field are positioned when said fields are directed onto said exposure area, wherein the apparatus comprises a digital camera arranged to generate an image of the scale and the X-ray indicating element when the X-ray indicating element emits light due to an exposure of said X-ray field.

7. An apparatus according to claim 6, wherein the camera is a digital video camera.

8. An apparatus according to claim 6, wherein the camera is fixed to a flexible arm that allows the position of the camera to be adjusted.

9. An apparatus according to claim 6, comprising at least one marker for indicating on the scale the position of the edge of the light field, wherein said at least one marker is X-ray detectable.

10. An apparatus according to claim 6, comprising means for allowing visualization of the image or images recorded by the camera.

11. Device for determining alignment of a light field and an X-ray field of an X-ray apparatus, said device comprising
an X-ray indicating element configured to emit light upon exposure to X-rays in such a way that parts exposed to X-rays can be distinguished from non-exposed parts, and
a visible scale arranged in association with the X-ray indicating element such as to allow determination of where on the scale an edge of the light field and an edge of the X-ray field are positioned, wherein the device comprises a first and a second unit, wherein each of said units comprises a set of said X-ray indicating element and scale, and wherein said first and second units are rotatably connected to each other.

12. Device according to claim 11, comprising four units, each of which have an elongated shape and comprise a set of said X-ray indicating element and scale, wherein said four units are rotatably connected to each other such as to allow formation of a cross and to allow positioning on top of each other.

13. Device according to claim 11, wherein the X-ray indicating element is capable of fluorescing upon exposure to X-rays.

14. Device according to claim 13, wherein the X-ray indicating element comprises Gd2O2S:Tb.

15. Device according to claim 11, wherein each unit comprises an X-ray detectable scale.
